(19) United States
(12) Patent Application Publication
(10) Pub. No.: US 2017/0071526 A1
(43) Pub. Date: Mar. 16, 2017

(54) Mouthguard that Detects a Concussive Impact

(71) Applicant: Philip Scott Lyren, Bangkok (TH)

(72) Inventor: Philip Scott Lyren, Bangkok (TH)

(21) Appl. No.: 14/850,972

(22) Filed: Sep. 11, 2015

(51) Int. Cl. A61B 5/00; A61B 5/103; A63B 71/08

(52) U.S. Cl. CPC A61B 5/4064; A63B 71/085; A61B 5/1032; A61B 5/0048

(57) ABSTRACT

A mouthguard detects when a wearer of the mouthguard receives a concussive impact. The mouthguard includes a U-shaped body and a concussion detection mechanism that undergoes a chemical or physical reaction when the wearer receives the concussive impact.

### BACKGROUND

Thousands of sports-related concussions occur in the United States each year. Many of these concussions happen in high school, college, and professional contact sports, but young kids playing a contact sport can also experience a concussion.

When an injury to the brain occurs from an impact or blow to the head, the recipient of the blow should immediately discontinue playing the sport and consult a health-care professional as soon as possible. Unfortunately, injuries to the brain from a concussive blow are not readily observable to the person receiving the blow or other people witnessing the impact.

Advancements in devices that can detect when a person receives a concussive blow will reduce brain injuries associated with concussions.

### BRIEF DESCRIPTION OF THE DRAWINGS

FIG. 1A is a mouthguard with a concussion detection mechanism that has not been activated according to an example embodiment.

FIG. 1B is the mouthguard of FIG. 1A in which the concussion detection mechanism has been activated according to an example embodiment.

FIG. 2A is a mouthguard with three concussion detection mechanisms that have not been activated (a channel inside the mouthguard connects the concussion detection mechanisms) according to an example embodiment.

FIG. 2B is the mouthguard of FIG. 2A in which the three concussion detection mechanisms have been activated according to an example embodiment.

FIG. 3A is a mouthguard with a U-shaped concussion detection mechanism that has not been activated according to an example embodiment.

FIG. 3B is the mouthguard of FIG. 3A in which the U-shaped concussion detection mechanism has been activated according to an example embodiment.

FIG. 4A is a mouthguard with an X-shaped concussion detection mechanism that has not been activated according to an example embodiment.

FIG. 4B is the mouthguard of FIG. 4A in which the X-shaped concussion detection mechanism has been activated according to an example embodiment.

FIG. 5 is a mouthguard with a cylindrical-shaped concussion detection mechanism that fits into the body of the mouthguard according to an example embodiment.

FIG. 6 is a mouthguard with three rectangular-shaped concussion detection mechanisms that fit into the body of the mouthguard according to an example embodiment.

FIG. 7 is a cylindrical-shaped concussion detection mechanism with a membrane that separates two cavities having an agent before the concussion detection mechanism is activated according to an example embodiment.

FIG. 8 is a U-shaped concussion detection mechanism with a membrane that separates two cavities having an agent before the concussion detection mechanism is activated according to an example embodiment.

FIG. 9 is a rectangular-shaped concussion detection mechanism with a vertical middle-membrane that separates two cavities having an agent before the concussion detection mechanism is activated according to an example embodiment.

FIG. 10 is a rectangular-shaped concussion detection mechanism with a horizontal middle-membrane that separates two cavities having an agent before the concussion detection mechanism is activated according to an example embodiment.

FIG. 11 is a concussion detection mechanism with honeycomb-shaped cells before the concussion detection mechanism is activated according to an example embodiment.

FIG. 12 is a concussion detection mechanism with bubble-shaped cells before the concussion detection mechanism is activated according to an example embodiment.

FIG. 13A is a person wearing a football helmet with a mouthguard before a concussion detection mechanism in the mouthguard is activated from a concussive impact according to an example embodiment.

FIG. 13B is the person of FIG. 13A in which the concussion detection mechanism in the mouthguard is activated from a concussive impact according to an example embodiment.

FIG. 14 is a method to indicate in a mouthguard when a wearer of the mouthguard receives a concussive impact according to an example embodiment.

### SUMMARY OF THE INVENTION

One example embodiment is a mouthguard that detects when a wearer of the mouthguard receives a concussive impact. The mouthguard includes a U-shaped body and a concussion detection mechanism that undergoes a reaction when the wearer receives the concussive impact.

Other example embodiments are discussed herein.

### DETAILED DESCRIPTION

Example embodiments include methods and apparatus that detect with a mouthguard when a person wearing the mouthguard receives a concussive blow or concussive impact. People wear the mouthguard inside their mouth to protect themselves against injury. For instance, the mouthguard functions to protect one or more of the lips, mouth, teeth, gums, tongue, and head of a wearer when the wearer receives an impact or blow (such as the wearer receiving a blow while playing a contact sport like football, hockey, or lacrosse).

The mouthguard has a body with a U-shape that is sized to fit inside a mouth of the wearer. The body includes a base and one or more walls that extend outwardly from the base. The base follows a contour of the jaw of the wearer and abuts against a bottom side of the teeth (i.e., the bite side of the teeth), and one or more of the walls follow the contour of the jaw and abut against a front or back side of the teeth. When the mouthguard is being worn, teeth of the wearer abut against the base and walls so the mouthguard snuggly and securely fits inside the mouth of the wearer.

The concussion detection mechanism is positioned at one or more locations in, at, or with the body of the mouthguard. For example, the concussion detection mechanism attaches to or engages with a surface of and/or interior portion of the body, integrally forms into or with the body, embeds into the body, or is surrounded by the body (such as being partially surrounded by the body or completely surrounded on all of its sides by the body).

The concussion detection mechanism activates when a wearer of the mouthguard receives a single concussive impact or multiple impacts that have an accumulative effect to render a concussion. By way of example, the concussion detection mechanism includes, but is not limited to, one or more of an insert (such as a body that is separate and distinct from the body of the mouthguard), a cavity or opening formed in the body of the mouthguard, a channel or passageway formed in the body of the mouthguard, and a portion or section of the body of the mouthguard formed of a different material than other portions of the body. Furthermore, the insert can be a device that detects a concussive blow and activates upon receiving a concussive blow using one or more of electronic or electrical components, mechanical components, and chemical components.

In an example embodiment, a front and/or sides of the mouthguard include a concussion detection mechanism with an insert or cavity that is filled with or includes a color-changing chemical, gas, or material or a deformable or breakable material or another material discussed herein. Further, the concussion detection mechanism can include a material or substance that changes one or more of its properties upon receiving an impact. For example, the concussion detection mechanism initially has a color (such as being green, blue, or clear) that signifies a wearer of the mouth has not received a concussive impact. When the wearer of the mouthguard experiences an impact large enough to generate a concussion or possibly generate a concussion, the concussion detection mechanism bursts, breaks, transforms, or undergoes a physical reaction and/or chemical reaction to change color (such as changing to the color red). This change in color changes the color of the mouthguard and provides a clear visible indication that the wearer experienced a concussive impact.

In an example embodiment, the concussion detection mechanism includes an insert, cavity, and/or material that bursts, breaks, and/or changes when a head of the wearer experiences an impact sufficient to generate a concussion or possible concussion in the wearer. The insert, cavity, and/or material do not change when the wearer receives an impact that is insufficient to produce a concussion or possible concussion in the wearer. Furthermore, one or more portions of the concussion detection mechanism are located in the body of the mouthguard such that third parties can see the concussion detection mechanism when the mouthguard is inserted in the mouth of the wearer. For instance, when a player wearing the mouthguard experiences a concussive impact, other players, a coach, or the player himself can see the change in color or other change of the mouthguard. Upon seeing the change, a coach would take a player out of the game. Players would not be allowed to play unless their mouthguards were visibly clear or showing a “go” color or “go” indicia.

In one embodiment, the mouthguard is formed of or includes a pliable polymeric body (such as a body formed of polyethylene or another polymer).

In an example embodiment, the concussion detection mechanism includes a device or an insert that fits inside the mouthguard and is not removable or accessible (i.e., no one can tamper with the insert without destroying the mouthguard and/or activating the insert or material or cavity where the insert is located). For example, the insert includes two or more separate chambers or cells that are separated by a thin plastic, polymeric membrane. When the mouthguard experiences an impact significant enough to result in a concussion, the membrane breaks and the two liquids contact each other and cause a physical reaction and/or chemical reaction to occur in which the liquids change color. The liquids, now a visible color, fill the insert and/or cavity and can be readily seen.

In some instances, a concussion can result from a single strong impact to the head if the force of the impact is sufficient to rise to a level of a concussive impact. In other instances, however, successive smaller impacts can cause a concussion even though the force of each impact alone may not be sufficient to rise to the level of a concussive impact. Successive impacts to the head can be accumulative and result in a concussion. Furthermore, a second concussive impact soon after a first concussive impact can be relatively weak compared to the first concussive impact but can nonetheless effect a severe concussion to the receiver of the impacts. Example embodiments include mouthguards that detect and activate after receiving two or more smaller impacts that individually would not result in a concussion but accumulatively would result in a concussion.

In an example embodiment, each time the mouthguard receives an impact the insert, cavity, or material weakens if an accumulative effect of such impacts would result in a concussion. Each time the mouthguard receives such an impact, a structural integrity or strength of the insert, cavity, or material diminishes or degrades. When the accumulative effect of the impacts results in a concussion, then the mouthguard activates (e.g., the insert breaks, the cavity bursts, the material changes or transforms, etc.). This activation provides a visual or perceivable indication (e.g., with one of the five senses) that the wearer received enough accumulative impacts to result in a concussion or a possible concussion.

The mouthguard can detect and activate upon receiving multiple impacts in other ways as well. Consider an example in which the insert or cavity includes a plurality of smaller cells (such as smaller inserts, cavities, or material portions or locations). Each cell is separate and independent from another cell. For example, each cell is sealed from an adjacent cell, such as being hermetically sealed, airtight, fluid tight, and/or watertight. Upon receiving an impact, one or more cells activate. An impact with a larger force induces more cells to activate when compared to an impact with a relatively smaller force. For instance, a number of cells that activate is proportionate to a size or magnitude of an impact.

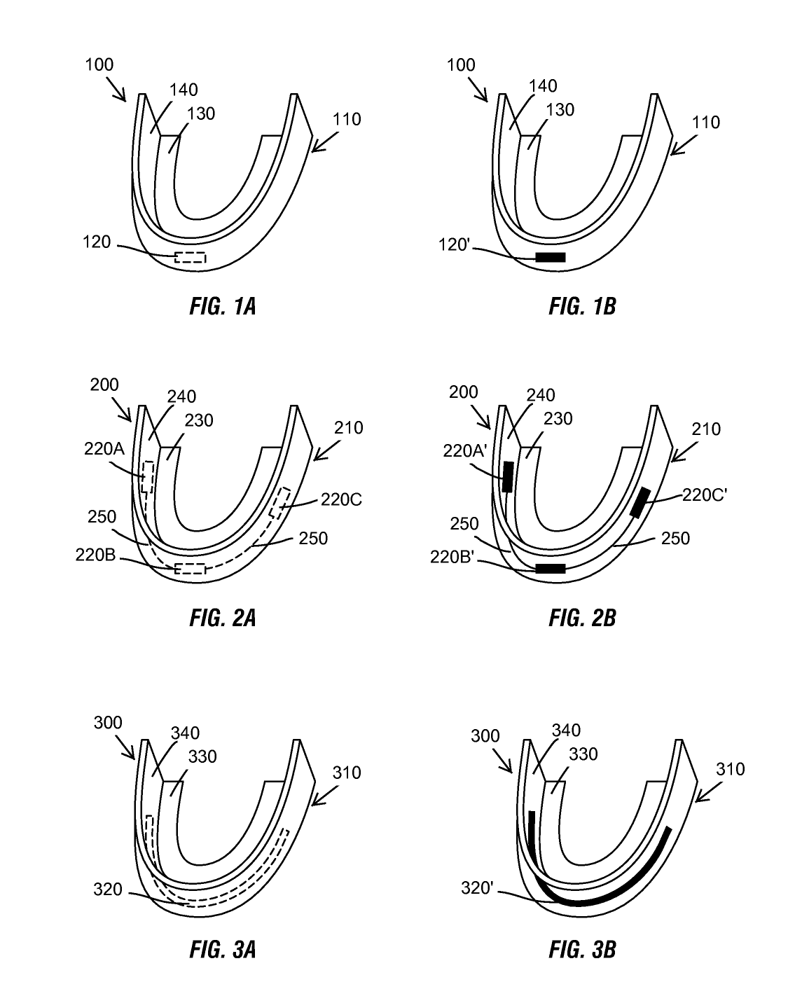

FIG. 1A is a mouthguard 100 with a body 110 that includes a concussion detection mechanism 120 that has not been activated according to an example embodiment. The body 110 includes a U-shaped base 130 and a U-shaped front wall 140 that extends outwardly from the base 130. The concussion detection mechanism 120 is shown with dashed lines to signify that it is fully or partially embedded in or attached to the body 110 and/or located behind the front wall 140.

The mouthguard 100 is sized to fit inside a mouth of a wearer. The base 130 and front wall 140 follow a contour of the jaw of the wearer with the front wall 140 abutting a front side of the teeth of the wearer and the base 130 abutting a down side of the teeth (such as when the wearer bites down on the base 130 of the mouthguard 100).

The concussion detection mechanism 120 is located at or in the front wall 140. For example as shown in FIG. 1A, the concussion detection mechanism 120 is embedded inside of and surrounded by the body of the front wall 140.

FIG. 1B shows the mouthguard 100 in which the concussion detection mechanism 120′ has been activated according to an example embodiment (a prime 0 being used to indicate activation of a concussion detection mechanism).

Activation of the concussion detection mechanism changes a physical, chemical, and/or electrical state of the concussion mechanism so this activation is visible or perceivable with one of the five senses to the wearer and/or third parties viewing the wearer with the mouthguard. By way of example, the concussion detection mechanism changes color or produces a visible color when it is in the activated state. Activation can also include changes associated with a chemical reaction or a physical reaction (also known as a physical change).

When a wearer of the mouthguard receives a concussive blow, the concussion detection mechanism activates and changes from an un-activated state (shown in FIG. 1A as 120) to an activated state (shown in FIG. 1B as 120′).

When the concussion detection mechanism is in the un-activated state, it may be invisible or not detectable to the wearer and/or third parties. Alternatively, while in this state, the concussion detection mechanism can be partially or fully visible. Regardless of the visibility, the wearer and/or third parties can view the mouthguard and visually determine or otherwise perceive the state of the concussion detection mechanism.

The concussion detection mechanism is partially or fully visible to the wearer and/or third parties when it is in the activated state. By way of example, the concussion detection mechanism undergoes, produces, generates, or transforms a change in color (such as changing or adding a color red) or a change in its physical or material property. For instance, the wearer and/or third parties can view the mouthguard while in the mouth of the wearer and visually determine that the wearer experienced a concussive impact.

In an example embodiment, the concussion detection mechanism is located at a front or tip of the U-shaped front wall (or a bottom wall if the mouthguard has such a wall) such that third parties can see when the concussion mechanism is activated. Alternatively or additionally, the concussion detection mechanism is located on a portion of the body of the mouthguard that is outside of the mouth of the wearer (i.e., exposed so third parties can see the concussion detection mechanism when the wearer wears the mouthguard in his or her mouth).

Consider an example in which a wearer wears the mouthguard 100 shown in FIG. 1A while playing a contact sport, such as football. While in the un-activated state, the concussion detection mechanism 120 remains clear or not visible while embedded inside of the mouthguard. During a game, however, the wearer experiences a concussive blow to his head. An impact of this blow causes the concussion detection mechanism to transform or change from being clear or not visible (as shown in FIG. 1A being outlined with dashed lines) to be visible or activated (as shown in FIG. 1B being filled with solid black). The wearer is unaware that he received a blow significant enough to produce a concussion or possible concussion and intends to continue playing football. Another player or coach, however, notices that the concussion detection mechanism in the mouthguard is activated. The wearer is notified and he immediately stops playing the game and seeks aid from a medical professional to determine the extent of his concussion.

FIG. 2A is a mouthguard 200 with a body 210 that includes three separate concussion detection mechanisms 220A, 220B, and 220C that have not been activated according to an example embodiment. The body 210 includes a U-shaped base 230 and a U-shaped front wall 240 that extends outwardly from the base 230. A channel or pathway 250 inside the mouthguard connects the concussion detection mechanisms 220A, 220B, and 220C. The concussion detection mechanisms 220A, 220B, and 220C and channel 250 are shown with dashed lines to signify that they are fully or partially embedded in or attached to the body 210 and/or located behind the front wall 240.

By way of example, the channel 250 provides one or more of an electrical pathway or connection between the concussion detection mechanisms (such that the concussion detection mechanisms are in electrical communication with each other), a fluid pathway or connection between the concussion detection mechanisms (such that the concussion detection mechanisms are in fluid communication with each other), and a gas pathway or connection between the concussion detection mechanisms (such that the concussion detection mechanisms are in gaseous communication with each other).

As shown in FIG. 2A, one concussion detection mechanism 220A is located in one leg or side of the U-shaped front wall 240; one concussion detection mechanism 220B is located in a front portion or front side of the U-shaped front wall 240; and one concussion detection mechanism 220C is located in one leg or side of the U-shaped front wall 240 (oppositely disposed from the concussion detection mechanism 220A). The channel 250 extends from the first concussion detection mechanism 220A to the second concussion detection mechanism 220B, and from the second concussion detection mechanism 220B to the third concussion detection mechanism 220C. The channel can also extend to one or more exterior surfaces on the body 210 of the mouthguard.

FIG. 2B is the mouthguard 200 of FIG. 2A in which the three concussion detection mechanisms 220A′, 220B′, and 220C′ have been activated according to an example embodiment. When a wearer of the mouthguard receives a concussive blow, one or more of the concussion detection mechanisms activate and change from an un-activated state (shown in FIG. 2A as 220A, 220B, and 220C) to an activated state (shown in FIG. 2B as 220A′, 220B′, and 220C′).

In an example embodiment, all three of the concussion detection mechanisms activate when the wearer of the mouthguard receives a concussive blow or a blow that possible could result in a concussion.

In another example embodiment, one or more of the concussion detection mechanisms activate when the wearer of the mouthguard receives a concussive blow or a blow that possibly could result in a concussion. Activation of a selective one of the concussion detection mechanisms provides information to a medical professional and assists this professional in evaluating the wearer of the mouthguard after he or she receives an impact.

With reference to FIG. 2A, consider a reference frame in which a wearer wears the mouthguard 200 with the concussion detection mechanism 220A positioned at a right side of the jaw of the wearer, the concussion detection mechanism 220B positioned in a front of the jaw of the wearer, and the concussion detection mechanism 220C positioned at a left side of the jaw of the wearer. Activation of which concussion detection mechanism depends on which side of the head the wearer receives the impact. When the wearer receives the blow on the right side of the head, concussion detection mechanism 220A activates since it is located on the right side of the jaw. When the wearer receives the blow on the left side of the head, concussion detection mechanism 220C activates since it is located on the left side of the jaw. When the wearer receives the blow on the front side of the head, concussion detection mechanism 220B activates since it is located on the right side of the jaw. When the wearer receives the blow on the back of the head, all three concussion detection mechanisms 220A, 220B, and 220C activate. Here, a health care professional (such as a doctor or professional trained in concussion protocol) can readily determine where the wearer received the blow (i.e., to the right side of the head, to the left side of the head, to the front of the head, or to the back of the head). This information can assist in diagnosing and treating the recipient of the blow.

Activation of a selective one of the concussion detection mechanisms can be accomplished by varying mechanical or physical properties of one or more of the concussion detection mechanisms. Consider an example in which a concussion detection mechanism is designed to activate based on which side it receives the force of the impact. By way of example, this variation in activation is accomplished with one of varying a thickness of a wall or a side of a concussion detection mechanism, varying a thickness of a membrane inside a concussion detection mechanism, varying a shape, size, or angle of a concussion detection mechanism positioned in the mouthguard, varying a shape, size, or angle of a wall or side of a concussion detection mechanism, varying a chemical composition of an agent included in the cells or areas of the concussion detection mechanism, varying a shape, size, or angle of a membrane inside a concussion detection mechanism, varying one or more material properties of a wall, side, or membrane of a concussion detection mechanism, and varying one or more material properties of material adjacent to or abutting a wall, side, or membrane of a concussion detection mechanism (such as varying one of a thickness of a material, a strength of the material, a porosity of the material, a density of the material, a weight of the material, and a composition of the material).

Consider an example in which a mouthguard has two concussion detection mechanisms that are located on opposite sides of the mouthguard (such as one being located on one leg or wall of a U-shaped mouthguard and the other being located on the other leg or wall). These two concussion detection mechanisms connect to or communicate with a cavity, space, or window that is located in a front of the mouthguard (e.g., a part of the mouthguard that is visible to third parties while the wearer wears the mouthguard in his or her mouth). While participating in a contact sport, a player wearing the mouthguard experiences a concussive blow. This blow activates a single one of the two concussion detection mechanisms (i.e., one concussion detection mechanism activates from the blow while the other concussion detection mechanism does not activate). The activated concussion detection mechanism generates, produces, or releases a fluid that leaks or drains into the front cavity that is located in the front of the mouthguard. The fluid (now collected or appearing in the front cavity) produces a change in color of the front cavity or provides a change in color of the front cavity. This change in color visually signifies to the player and/or other people that can see the mouthguard that the player experienced a concussive blow.

Consider an example in which a mouthguard includes a concussion detection mechanism embedded inside of a body of the mouthguard such that the concussion detection mechanism is not accessible (e.g., a wearer of the mouthguard cannot access, touch, tamper, or remove the concussion detection mechanism without destroying or damaging the mouthguard). The concussion detection mechanism includes an insert that is filled with an agent (such as a liquid). When the wearer of the mouthguard receives a concussive blow, the insert breaks and causes the liquid to leak out from the insert. This liquid travels along or through one or more channels that extend from the insert to an external surface of a body of the mouthguard and generates a rancid or bitter taste in the mouth of the wearer. This taste signifies to the wearer that he or she received a concussive blow. Further, the bitter taste causes the wearer to spit out the mouthguard and renders it unusable since wearing the mouthguard would produce an unwanted or annoying taste in the mouth of the wearer.

FIG. 3A is a mouthguard 300 with a body 310 that includes a U-shaped concussion detection mechanism 320 that has not been activated according to an example embodiment. The body 310 includes a U-shaped base 330 and a U-shaped front wall 340 that extends outwardly from the base 330. The concussion detection mechanism 320 is shown with dashed lines to signify that it is fully or partially embedded in or attached to the body 310 and/or located behind the front wall 340.

FIG. 3B is the mouthguard 300 of FIG. 3A in which the U-shaped concussion detection mechanism 320′ has been activated according to an example embodiment.

As shown in FIGS. 3A and 3B, the concussion detection mechanism has a U-shape that follows the contour or shape of the body and front wall of the mouthguard. It can be formed as a single unit or several different sections, portions, or parts that are adjacent to each other. When a section of the concussion detection mechanism becomes activated, it is visible through the mouthguard.

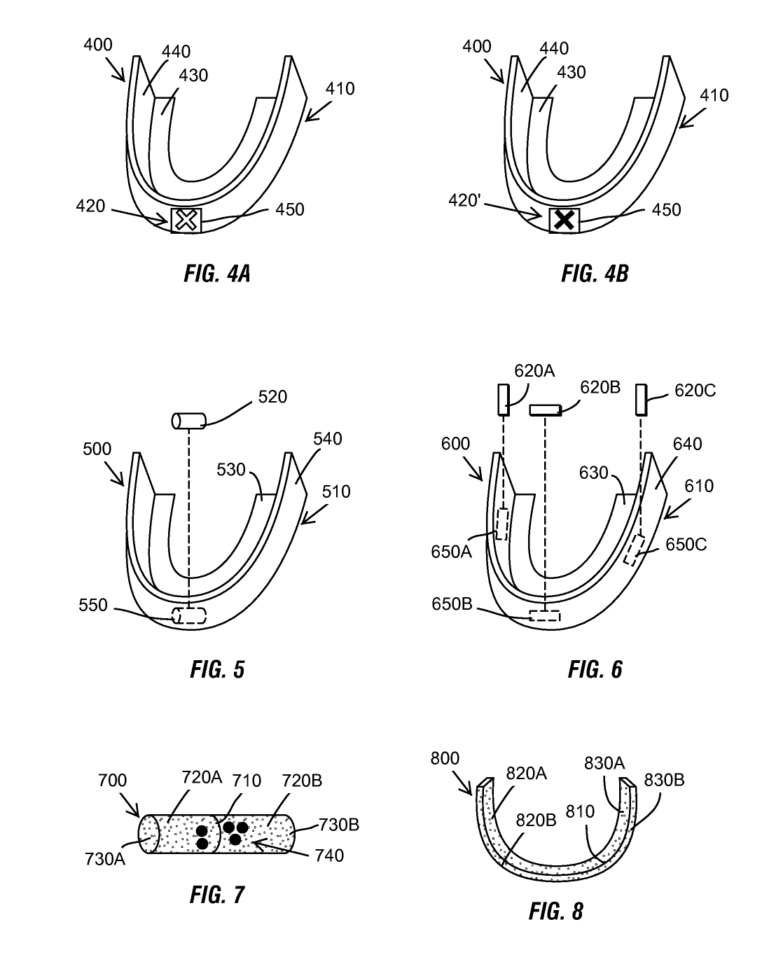

FIG. 4A is a mouthguard 400 with a body 410 that includes an X-shaped concussion detection mechanism 420 that has not been activated according to an example embodiment. The body 410 includes a U-shaped base 430 and a U-shaped front wall 440 that extends outwardly from the base 430. The concussion detection mechanism 420 is viewable within or through a window 450 located in the front wall 400.

FIG. 4B is the mouthguard 400 of FIG. 4A in which the X-shaped concussion detection mechanism 420′ has been activated according to an example embodiment.

As shown in FIGS. 4A and 4B, the concussion detection mechanism has an X-shape that is viewable through the window 450 embedded or located in the front wall 440. The window 450 can be an opening or clear cover within the front wall 440 or provide a frame or enclosure for the concussion detection mechanism. For example, the window is made from a translucent or transparent polymer or plastic.

Consider an example in which football players of a particular team wear orange mouthguards (orange being a color of the football team). The mouthguards include a square or rectangular window in the front of the mouthguard that is not colored orange in order to visibly distinguish the window from the rest of the mouthguard. For instance, the window has a different color other than orange, is clear, or is transparent. Behind or adjacent this window is a concussion detection mechanism that activates when a wearer of the mouthguard receives a concussive blow. During the game, the coach of the team, staff, officials, spectators, and/or other players can visible see the window of the mouthguard while the mouthguard is worn in the mouth of the players. During the game, two players collide and fall to the ground. When these players stand, an official notices that a concussion detection mechanism in one of the players has been activated. For example, the window in the mouthguard of this player is no longer clear but colored red. The official notifies the player who is escorted off the field to concussion evaluation team (e.g., a team of medical personal with expertise in evaluating and treating concussions).

FIG. 5 is a mouthguard 500 with a body 510 that includes a cylindrical-shaped concussion detection mechanism 520 that has not been activated according to an example embodiment. The body 510 includes a U-shaped base 530 and a U-shaped front wall 540 that extends outwardly from the base 530. The concussion detection mechanism 520 fits within a cavity 550 located inside the front wall 540.

FIG. 6 is a mouthguard 600 with a body 610 that includes three rectangular-shaped concussion detection mechanisms 620A, 620B, and 620C that have not been activated according to an example embodiment. The body 610 includes a U-shaped base 630 and a U-shaped front wall 640 that extends outwardly from the base 630. The concussion detection mechanisms 620A, 620B, and 620C fit within one of three corresponding cavities 650A, 650B, and 650C located inside the front wall 640.

In FIGS. 5 and 6, the concussion detection mechanisms are shown as inserts that are embedded within or attached to the body of the mouthguard. These inserts can be fabricated or molded into the body such that they are not removable or accessible without destroying the mouthguard.

Consider an example in which a concussion detection mechanism includes an agent as a dye or pigment. When the mouthguard receives a concussive impact, the concussion detection mechanism breaks or ruptures and the dye or pigment leaks or activates. Release or activation of the dye or pigment activates the concussion detection mechanism and signals that the wearer received a concussive blow.

Consider an example in which a concussion detection mechanism includes agents that include a universal indicator and a liquid being one of an acid or a base. When the mouthguard receives a concussive impact, the concussion detection mechanism breaks or ruptures and the universal indicator mixes with acid or the base. For example, mixing the universal indicator with an acid results in a color of red, pink, orange, or yellow; and mixing the universal indicator with a base results in a color of green, blue, or purple. Release or activation of the dye or pigment activates the concussion detection mechanism and signals that the wearer received a concussive blow.

The concussion detection mechanisms can have various shapes and sizes. By way of example, these shapes include, but are not limited to, a rectangular prism, a cube, a triangular prism, an octagonal prism, a cylinder, a tetrahedron, a pyramid, a cone, a sphere, and other three-dimensional objects. Some examples of these shapes are shown in FIGS. 7-12.

FIG. 7 is a cylindrical-shaped concussion detection mechanism 700 with a wall or membrane 710 that separates two chambers or cavities 720A and 720B before the concussion detection mechanism is activated according to an example embodiment.

Cavity 720A is filled with or includes an agent 730A (such as a material, an object, and/or a reactant), and cavity 720B is filled with or includes another agent 730B different than the first agent (such as material, an object, and/or a reactant). When the membrane 710 breaks or ruptures, the agents 730A and 730B generate a reaction (such as a chemical or physical reaction). This reaction activates the concussion detection mechanism 700 and provides people with visual proof or confirmation that the mouthguard experienced a concussive blow or potentially concussive blow.

By way of example, the membrane 710 is located in the middle of the cylinder and provides a fluid-tight and/or gas-tight seal between cavities 720A and 720B. When the membrane is compromised (such as being partially or fully damaged or destroyed), the agents 730A and 730B contact each other or are exposed to each other and generate the reaction. For example, when a mouthguard housing the concussion detection mechanism 700 receives a concussive blow, the membrane 710 breaks or cracks causing a fluid, gas, or material in one cavity to come into contact with a fluid, gas, or material in the other cavity.

In an example embodiment, the membrane 710 includes a thin wall that extends between and seals the two cavities 720A and 720B. The wall is formed of a polymer or other material that breaks upon receiving a force of a concussive blow. An amount of force needed to break the wall and hence cause the two agents to mix or contact each other, can be varied, changed, set, or adjusted based on a thickness of the membrane, shape of the membrane, size of the membrane, and material properties of the membrane.

When the mouthguard receives a concussive blow, a force of the impact causes the concussion detection mechanism to break and hence initiate the reaction. The concussion detection mechanism can include one or more devices or objects to assist in rupturing, breaking, damaging, cracking, or destroying the concussion detection mechanism.

As shown in FIG. 7, the concussion detection mechanism 700 includes one or more objects 740 that are located in cavities 720A and 720B. By way of example, these objects are shown as balls or beads (such as metal or plastic balls). When the mouthguard receives a concussive blow, these balls slam against the membrane 710 and break it. For example, an impact of the balls hitting or pushing against the membrane causes it to rupture. Alternatively or additionally, the balls slam against one or more walls or sides of the body of the cylindrically shaped concussion detection membrane and cause the wall or side to break or rupture.

Objects in a cavity are not limited to a ball or bead but include objects with other shapes (such as an object with another three-dimensional shape or an object with a sharp, pointed, or jagged edge).

Consider an example in which a mouthguard includes a concussion detection mechanism with a plastic insert that has two chambers. A wall separates and hermetically seals these two chambers from each other such that a first chamber houses or contains a first liquid and a second chamber houses or contains a second liquid. Each chamber also includes a plurality of metal balls. When a wearer of the mouthguard receives a concussive blow, the balls in the first chamber impact against the wall and cause it to crack. In response to this crack, the two fluids mix together (such as the first fluid draining or leaking into the second chamber and/or the second fluid draining or leaking into the first chamber). A physical or chemical reaction occurs when these two fluids mix together and the product of this mixture changes to a red color (or another color that is different than the color of either the first fluid or the second fluid). The red color of the fluid resulting from the physical or chemical reaction is visible through an opening or window in the mouthguard, and this color visually signifies to people that the person wearing the mouthguard experienced a concussive blow.

Consider an example in which a mouthguard includes a concussion detection mechanism with a three-dimensional plastic insert that fits inside of a cavity in the mouthguard. The cavity is larger than the insert. Further, the insert is filled with or includes a first reactant or agent, and the cavity is filled with or includes a second reactant or agent. As such, the second reactant surrounds an external surface of the insert (such as the insert being immersed in the second reactant). When the mouthguard receives a concussive impact, the insert strikes a wall of the cavity and ruptures. Rupturing of the insert causes the first reactant to mix with the second reactant and results in a physical or chemical reaction. For example, the cavity generates a light, glows, changes color, or produces a discernable change to signify that the wearer of the mouthguard received an impact sufficient to cause a concussion or possibly cause a concussion.

FIG. 8 is a U-shaped concussion detection mechanism 800 with a wall or membrane 810 that separates two chambers or cavities 820A and 820B before the concussion detection mechanism is activated according to an example embodiment.

Cavity 820A is filled with or includes an agent 830A (such as a material, an object, and/or a reactant), and cavity 820B is filled with or includes another agent 830B different than the first agent (such as material, an object, and/or a reactant). When the membrane 810 breaks or ruptures, the agents 830A and 830B generate a reaction (such as a physical or chemical reaction). This reaction activates the concussion detection mechanism 800 and provides people with visual proof or perceivable confirmation that the mouthguard experienced a concussive blow or potentially concussive blow.

Consider an example in which the mouthguard includes a U-shaped concussion detection mechanism that breaks or cracks upon receiving a force equivalent to a concussive blow in football. A visual inspection of the concussion detection mechanism reveals whether the wearer received a concussive blow (i.e., when the U-shaped member is cracked or broken, the wearer received such a blow).

Consider an example in which a mouthguard includes a U-shaped concussion detection mechanism that follows a contour of the mouthguard along a front wall. When a wearer of the mouthguard receives a concussive blow, the concussion detection mechanism bends and causes a wall or membrane to break or crack. This breakage, in turn, initiates a physical or chemical reaction between two reactants or agents housed in the concussion detection mechanism. A wearer of the mouthguard senses the physical or chemical reaction and determines, from this sensation, that he or she received a concussive blow. For example, the physical or chemical reaction causes the mouthguard to emit a perceivable scent or smell. As another example, the physical or chemical reaction causes the mouthguard to emit a light, glow, or change color. As another example, the physical or chemical reaction causes the mouthguard to emit a sound. As another example, the physical or chemical reaction causes the mouthguard to emit a bitter taste in the mouth of the wearer.

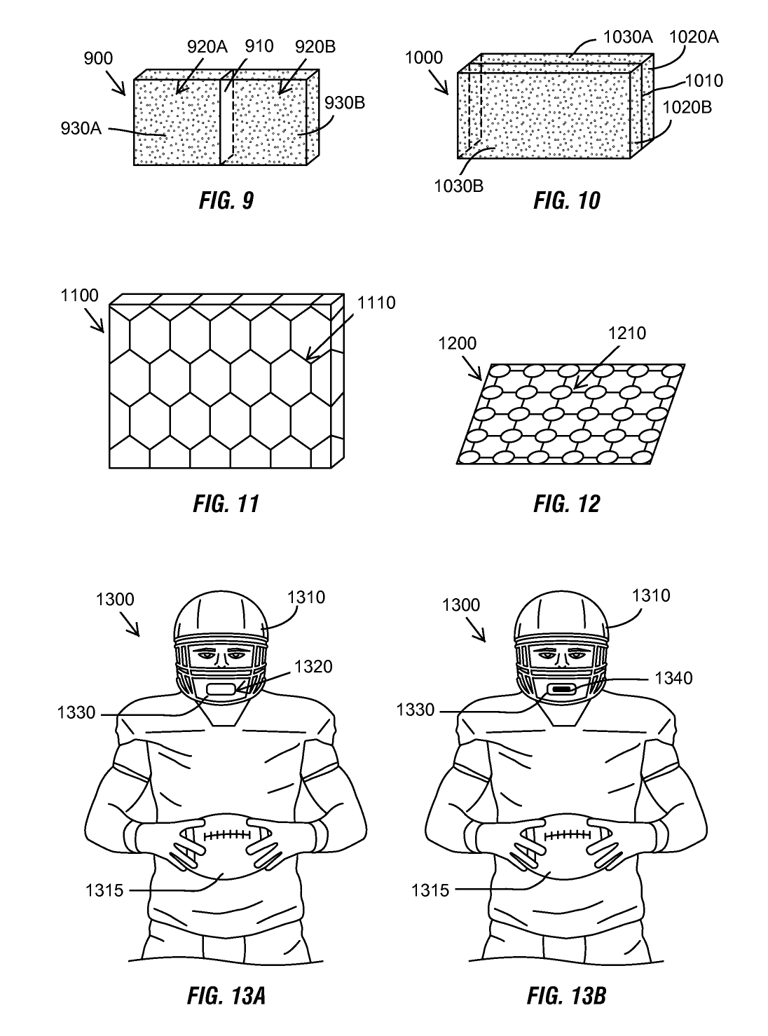

FIG. 9 is a rectangular-shaped concussion detection mechanism 900 with a vertically oriented wall or membrane 910 that separates two chambers or cavities 920A and 920B before the concussion detection mechanism is activated according to an example embodiment. The membrane 910 extends through a middle of the cavities and separates them into two equal or approximately equal halves.

Cavity 920A is filled with or includes an agent 930A (such as a material, an object, and/or a reactant), and cavity 920B is filled with or includes another agent 930B different than the first agent (such as material, an object, and/or a reactant). When the membrane 910 breaks or ruptures, the agents 930A and 930B generate a reaction (such as a physical or chemical reaction). This reaction activates the concussion detection mechanism 900 and provides people with visual proof or perceivable confirmation that the mouthguard experienced a concussive blow or potentially concussive blow.

FIG. 10 is a rectangular-shaped concussion detection mechanism 1000 with a horizontally oriented wall or membrane 1010 that separates two chambers or cavities 1020A and 1020B before the concussion detection mechanism is activated according to an example embodiment. The membrane 1010 extends through a middle of the cavities and separates them into two equal or approximately equal halves.

Cavity 1020A is filled with or includes an agent 1030A (such as a material, an object, and/or a reactant), and cavity 1020B is filled with or includes another agent 1030B different than the first agent (such as material, an object, and/or a reactant). When the membrane 1010 breaks or ruptures, the agents 1030A and 1030B generate a reaction (such as a physical or chemical reaction). This reaction activates the concussion detection mechanism 1000 and provides people with visual proof or perceivable confirmation that the mouthguard experienced a concussive blow or potentially concussive blow.

FIG. 11 is a concussion detection mechanism 1100 with honeycomb-shaped cells 1110 including, filled with, or surrounded by an agent before the concussion detection mechanism is activated according to an example embodiment.

FIG. 12 is a concussion detection mechanism 1200 with bubble-shaped cells 1210 including, filled with, or surrounded by an agent before the concussion detection mechanism is activated according to an example embodiment.

Consider an example in which a mouthguard includes a honey-combed shaped concussion detection mechanism and/or a bubble-shaped concussion detection mechanism that is embedded inside of and surrounded by a body of the mouthguard. When a wearer of the mouthguard receives a concussive blow, one or more of the cells in the honey-comb structure or bubble structure burst. A wearer of the mouthguard or another person looking at the mouthguard sees burst cells and is alerted that the wearer received a concussive blow.

Consider another example in which a mouthguard includes a honey-combed shaped concussion detection mechanism and/or or a bubble-shaped concussion detection mechanism that is embedded inside of and surrounded by a body of the mouthguard. The honey-combed structure and/or bubble structure is located away from the base of the mouthguard such that when the wearer bites down on the mouthguard, none of the cells of the structure break. When a wearer of the mouthguard receives a concussive blow, however, one or more of the cells in the structure burst. The broken or burst cells form a pattern that indicates a magnitude and direction of the impact. For example, more cells are broken on a left side of the structure, and this indicates that the wearer received a blow from the left side. Additionally, a number of burst cells indicate a magnitude of the blow (such as a smaller number of burst cells indicating a concussive blow of lesser magnitude than a larger number of burst cells).

Consider another example in which a mouthguard includes a honey-combed shaped concussion detection mechanism and/or or a bubble-shaped concussion detection mechanism that is embedded inside of and surrounded by a cavity located in a body of the mouthguard. The cavity includes a first reactant, and the cells include a second reactant. When the mouthguard receives a concussive blow, one or more of the cells break. This breakage causes the first and second reactants to mix and produce a reaction (such as a physical, chemical, or electrochemical reaction).

Mouthguards can be made in various shapes and sizes and are not limited to U-shaped body. For example, some mouthguards include a round, oval, or rectangular member that connects to a main body via an arm, wing, or extension. This member covers the lips of a wearer while the base of the mouthguard is inserted into the mouth of the wearer. FIGS. 13A and 13B show an example of such a mouthguard that is often worn by football players (such as college and professional football players in America).

FIG. 13A is a person 1300 wearing a football helmet 1310 with a mouthguard 1320 before a concussion detection mechanism in the mouthguard is activated from a concussive impact according to an example embodiment. For illustration, the person is shown as a football player wearing a football uniform and holding a football 1315.

The mouthguard 1320 includes a round, oval, or rectangular-shaped extension 1330 that extends outwardly from a U-shaped body (see FIG. 5 for an example of a U-shaped body). When the person 1300 inserts the mouthguard 1320 into his mouth and bites down on it, the extension 1330 extends out from his mouth and covers his lips. In this manner, the extension provides a protective shield or barrier to the mouth of the person.

FIG. 13B is the person 1300 of FIG. 13A in which a concussion detection mechanism 1340 in the extension 1330 of the mouthguard 1320 is activated from a concussive impact according to an example embodiment.

Consider an example in which football players wear the mouthguard shown in FIGS. 13A and 13B. Each mouthguard includes a concussion detection mechanism located in the extension that covers the mouth of the wearer. During a tackle, two players experience helmet-to-helmet contact that results in a violent blow. A concussion detection mechanism in the mouthguards of these two players activates. A referee sees that the concussion detection mechanisms were activated since they are clearly visible on or through the extensions of the mouthguards.

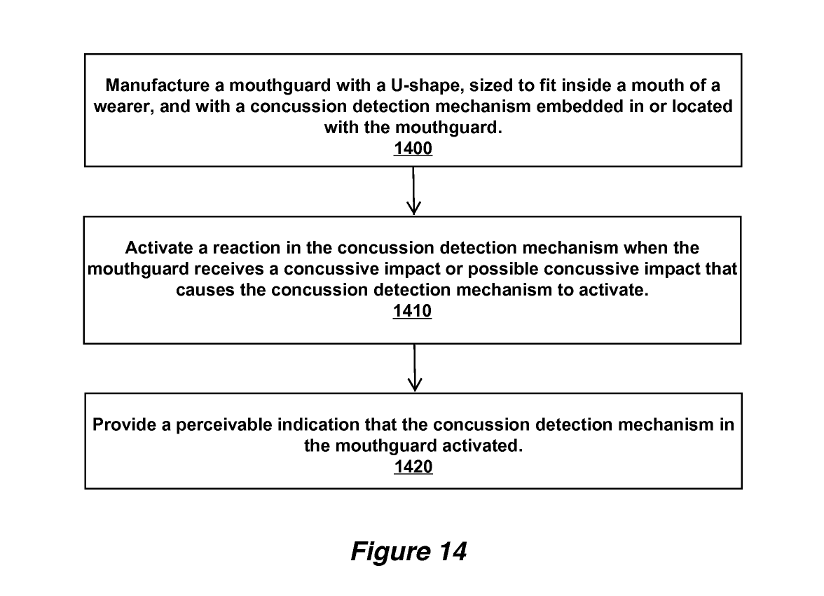

FIG. 14 is a method to indicate in a mouthguard when a wearer of the mouthguard receives a concussive impact according to an example embodiment.

Block 1400 states manufacture a mouthguard with a U-shape, sized to fit inside a mouth of a wearer, and with a concussion detection mechanism embedded in or located with the mouthguard.

Mouthguard can be made from a variety of materials, such as poly (vinyl acetate-ethylene) copolymer clear thermoplastic or thermoplastic material, polyurethane, laminated thermoplastic, and other polymers. For example, mouthguards can be made with an injection molding process or molding process, a vacuum-formed appliance, a pressure-laminated appliance, or other technique.

Block 1410 states activate a reaction in the concussion detection mechanism when the mouthguard receives a concussive impact or possible concussive impact that causes the concussion detection mechanism to activate. Examples of a reaction include, but are not limited to, a physical reaction, a chemical reaction, and an electrochemical reaction (i.e., process caused by or accompanied by passage of an electric current). Reactions also include deformation of a material or breaking of a material.

Block 1420 states provide a perceivable indication that the concussion detection mechanism in the mouthguard activated. For example, provide an indication that can be sensed or perceived by the wearer of the mouthguard or another person that views or examines the mouthguard (such as generating an indication perceivable from one or more of the five senses of sense, sound, sight, touch, smell, and taste). These indications includes, but are not limited to, one or more of changing a color of the mouthguard, causing the mouthguard to glow or emit light, causing taste to appear in a mouth of a wearer of the mouthguard, causing the mouthguard to undergo a physical or chemical reaction in which one or more reactants generate or produce a visible or perceivable product, causing the mouthguard to break, crack, deform, or become unusable, and causing the mouthguard change or alter one or more of its physical or material properties.

FIGS. 1-6 illustrate the concussion detection mechanism located at or in a front wall of the mouthguard. The concussion detection mechanism, however, can be located in other portions of the body of the mouthguard as well in accordance with an example embodiment.

In an example embodiment, the mouthguard and/or concussion detection mechanism includes one or more electronic components that provide detection and notification of a concussive blow. These electronic components include, but not limited to, a chip, a printed circuit board with electrical components, an accelerometer, piezoelectric sensor, impact force sensor, a battery or power supply, a transmitter, a receiver, a transceiver, a memory, and a processor.

In an example embodiment, the mouthguard and/or concussion detection mechanism does not include one or more electronic components. Such electronic components increase a cost of manufacturing the mouthguard, and a lower priced mouthguard is more affordable, especially to children, parents, and teams with a limited budget. Further, a lower costing mouthguard can be disposable and replaced with a new mouthguard at a lower price. Such a mouthguard, for example, includes a concussion detection mechanism that activates with a physical or chemical reaction or change in material property of the mouthguard. A mouthguard with no or limited electrical or moving parts is less expensive to manufacture than a mouthguard with electrical or moving parts.

Consider an example in which the concussion detection mechanism includes a deformable material that deforms with stress from a blow with a magnitude equivalent to blows received in contact sports. For example, compressive stress from the concussive blow deforms or shortens an object in the concussion detection mechanism and/or expands it outwardly. This movement, in turn, results in a cracked or broken concussion detection mechanism that people can view or perceive (e.g., a wearer hearing a break of the concussion detection mechanism from the impact or a person seeing that the concussion detection mechanism is cracked or broken).

The concussion detection mechanism can undergo a chemical reaction and/or physical reaction (also known as a physical change). A difference exists between a chemical reaction and a physical reaction. In a chemical reaction, a change occurs in the composition of the substance or substances in question. A chemical reaction results in the formation of a new chemical substance. For example, bonds are broken and new bonds are formed as a chemical change occurs. By contrast, in a physical reaction, a change does not occur to the composition but a difference occurs with another aspect of the substance, such as a change to a physical property, such as smell, color, or appearance, for example. Thus, a physical reaction can rearrange molecules but does not affect their internal structures.

Both physical and chemical reactions can result in perceivable indications. For example, indicators of a chemical change include a change in temperature, a change in color, a change in odor, the formation of bubbles, or the formation of a precipitate. As noted, indicators of a physical change include a change in texture, color, temperature, shape, state, et al.

Further, although the term “physical reaction” is used in the art, no reaction actually occurs since no change in elemental composition of the substance or substance occurs in a physical reaction. Reactions can include both a chemical reaction and a physical reaction (also known as a physical change).

Consider an example in which a concussion detection mechanism uses a starch indicator that turns dark blue or black to indicate a presence of a triiodide when a mouthguard experiences a concussive force. Consider another example in which a concussion detection mechanism uses a universal indicator that generates a color to indicate the presence of an acidic or alkaline solution. Consider another example in which a concussion detection mechanism uses one or more of the following to activate and indicate the mouthguard received a concussive blow: piezoelectric sensor (generating a voltage upon impact of the concussive blow), pH-sensitive polymers (a volume of the material changes when exposed to a change in PH of a surrounding medium), halochromic material (changing color upon contact with a substance that changes acidity), temperature-sensitive dyes, and photochromic materials (changing color in response to light) to name a few examples.

Example embodiments can use various agents to indicate activation of the concussion detection mechanism. Such agents include non-toxic substances that can be safely used in a mouthguard.

As used herein, a “chemical reaction” is a process that transforms one set of chemical substances to another. The substance or substances (a.k.a. reactants or reagents) are usually characterized by a chemical change and yield a product with one or more properties different from the reactants.

As used herein, a “concussion” is an injury to the brain that results in temporary or permanent loss of normal brain function. A concussion is usually caused by an impact to the head and may or may not include external signs of head trauma. Some people assume that concussions involve a loss of consciousness, but this assumption is not true. In many cases, a person with a concussion never loses consciousness. A concussion alters the way a brain functions, and the effects can include one or more of headaches, confusion, ringing in ears, nausea, vomiting, dizziness, blurred vision, blurred speech, and problems associated with judgment, memory, concentration, balance, and muscle coordination.

As used herein, a “concussive blow” or a “concussive impact” is a blow or impact that results in a concussion to the person receiving the blow or impact.

As used herein, a “concussion detection mechanism” is a mechanism that detects when a wearer receives a one or more impacts that result in a concussion or possibly result in a concussion.

As used herein, a “mouthguard” is a protective device that a person wears inside the mouth to prevent injury to the lips, mouth, teeth, gums, and/or head. The mouthguard covers the teeth and often the gums and is often worn by participants in a contact sport. The mouthguard is also called a mouth guard, a mouth piece, a mouthpiece, a mouth protector, or a gumguard.

As used herein, a “physical reaction” is a physical change that results in a difference in display without changing the composition of the substance or substances. Example changes in a physical reaction are changes to physical properties, such as color, texture, temperature, shape, state, luster, viscosity, solubility, volume, mass, and smell.

What is claimed is:

1. A mouthguard that activates when a wearer of the mouthguard receives a concussive impact, comprising:
a body that is formed of a polymer with a U-shaped base and a U-shaped front wall that extends outwardly from the base; and
a concussion detection mechanism that is embedded inside the front wall and includes a liquid that undergoes a chemical reaction and changes color when the wearer of the mouthguard receives the concussive impact.

2. The mouthguard of claim 1, wherein the concussion detection mechanism includes a plurality of cells in a honeycomb shape.

3. The mouthguard of claim 1, wherein the concussion detection mechanism includes a plurality of bubble-shaped cells.

4. The mouthguard of claim 1, wherein the concussion detection mechanism includes a cylinder that contains the liquid.

5. The mouthguard of claim 1, wherein the concussion detection mechanism includes a rectangular prism that contains the liquid.

6. The mouthguard of claim 1, wherein the concussion detection mechanism has a U-shape that contains the liquid.

7. The mouthguard of claim 1, wherein the concussion detection mechanism includes two cavities that contain the liquid and are separated from each other inside of the front wall.

8. A mouthguard that inserts into a mouth of a wearer and detects when the wearer of the mouthguard receives a concussive impact, comprising:
a U-shaped body that is sized to fit inside the mouth of the wearer and that includes a front wall that abuts against teeth of the wearer; and
a concussion detection mechanism that is enclosed within and surrounded by the front wall and that includes a liquid agent that undergoes a physical reaction and changes color when the wearer of the mouthguard receives the concussive impact.

9. The mouthguard of claim 8, wherein the concussion detection mechanism includes two cavities that enclose the agent and includes a membrane that separates the two cavities and breaks when the wearer of the mouthguard receives the concussive impact.

10. The mouthguard of claim 8, wherein the concussion detection mechanism includes a housing that holds the agent and that breaks and causes the physical reaction to occur when the wearer of the mouthguard receives the concussive impact.

11. The mouthguard of claim 8, wherein the concussion detection mechanism includes an elongated plastic insert that holds the agent and that is embedded inside of the front wall.

12. The mouthguard of claim 8, wherein the concussion detection mechanism includes two plastic inserts that store the agent and that are separated from each other.

13. The mouthguard of claim 8, wherein the concussion detection mechanism includes a channel that provides fluid communication between two cavities that are separated from each other in the front wall.

14. The mouthguard of claim 8, wherein the concussion detection mechanism includes an X-shaped cavity or X-shaped insert that changes color when the wearer of the mouthguard receives the concussive impact.

15. A method to indicate in a mouthguard when a wearer of the mouthguard receives a concussive impact, comprising:
manufacturing the mouthguard with a U-shape and sized to fit inside a mouth of the wearer and with a concussion detection mechanism embedded in a front wall of the mouthguard; and
activating one of a physical reaction or a chemical reaction in the concussion detection mechanism to change a color of the mouthguard when the mouthguard receives the concussive impact that causes the concussion detection mechanism to rupture.

16. The method of claim 15 further comprising:
flowing a liquid out of the concussion detection mechanism and through a channel in the mouthguard when the mouthguard receives the concussive impact that causes the concussion detection mechanism to rupture.

17. The method of claim 15 further comprising:
activating a plurality of cells in the concussion detection mechanism to change color when the mouthguard receives the concussive impact that causes the concussion detection mechanism to rupture.

18. The method of claim 15 further comprising:
changing a color of the mouthguard to red in response to the physical reaction or the chemical reaction when the mouthguard receives the concussive impact that causes the concussion detection mechanism to rupture.

19. The method of claim 15 further comprising:
mixing two liquids together in response to the physical reaction or the chemical reaction when the mouthguard receives the concussive impact that causes the concussion detection mechanism to rupture.

20. The method of claim 15 further comprising:
rupturing a plurality of liquid-filled cells in the concussion detection mechanism in response to the physical reaction or the chemical reaction when the mouthguard receives the concussive impact that causes the concussion detection mechanism to rupture.
